(19) United States
(12) Patent Application Publication
(10) Pub. No.: US 2022/0061662 A1
(43) Pub. Date: Mar. 3, 2022

(54) Mobile Ophthalmic Unit

(71) Applicant: VITALY SAMSONOV, LEVITTTOWN, PA (US)

(72) Inventor: VITALY SAMSONOV, LEVITTTOWN, PA (US)

(21) Appl. No.: 17/010,913

(22) Filed: Sep. 3, 2020

(51) Int. Cl. A61B 3/18; A61B 3/12; A61B 3/135; A61B 3/028

(52) U.S. Cl. CPC A61B 3/185; A61B 3/0285; A61B 3/135; A61B 3/12

(57) ABSTRACT

A mobile ophthalmic unit is disclosed. The apparatus is comprised of the following components: a phoropter on an adjustable arm, an extendable slit lamp, an ophthalmoscope with controller, desktop or a laptop computer (or both) with or without a detachable screen or a tablet computer, a WiFi router and an internet hotspot. All of the aforementioned components are connected to a mobile desk having locking caster wheels. An object of the invention is to allow an eye care specialist to visit patients in the field and perform routine eye exams remotely rather than requiring patients to travel in order to obtain eye care. For example, the ophthalmologist can easily roll the apparatus into a cargo van and drive to an underserved population and deliver eye exams where no such facilities exist—thereby enhancing eye care for a community.

### FIELD OF THE INVENTION

The invention relates to ophthalmology. More specifically, it relates to a mobile cart that allows sensitive ophthalmic equipment to travel to patients in the field.

### BACKGROUND

The history of ophthalmology dates back to the Bronze Age when the first written documentation regarding the eyes was recorded in 2250 B.C. In Egypt, the Ebers papyrus dating back to the year 1550 B.C. describes all the diseases and medical treatments that Egyptians were aware of the time. They knew about conditions such as cataracts, inflammation, granulation and dacryocystitis. While there is no evidence of surgery being performed at that time, history suggests that the Egyptian remedies worked. Much later in the 17th century Greeks performed couching for cataracts. This technique relied on the use of a sharp instrument, like a needle, where the eye is pierced at the edge of the cornea, and the opaque lens was pushed down so that light could enter the eye. It was not until the 18th and 19th centuries that further advances in ophthalmology were made possible by studying the eye's anatomy in detail. In the 1800s, Hermann von Helmholtz invented the ophthalmoscope which revolutionized the clinical study of the eye. This instrument allows eye professionals (including but not limited to ophthalmologists, optometrists or ophthalmic technicians) to see inside the fundus of the eye as part of a routine eye examination. It is crucial in determining the health conditions of the retina, optic disc and vitreous humor. The ophthalmoscope and further research in the field of ophthalmology opened the window for several eye doctors to subspecialize in particular diseases of the eye. Much later in the mid-1900s, Charles D. Kelman devised the cryo-probe for cataract extraction and Charles Schepens invented the indirect ophthalmoscope.

Today there are numerous devices for performing eye examinations. Some of the more popular equipment includes: Slit Lamp (a lighted microscope tool is what your doctor uses to look deep into the back of the eyes); Opthalmoscope (a small handheld machine used to check the retina, as well as the arteries and veins in your eye); Phoroptor (a large, plate with lenses that can be flipped back and forth to test the level of vision correction while the patient sits in an exam chair); and Keratometer is a dual-type of microscope that measures the cornea's shape. Many in the optometry industry have developed supports and work stations for such equipment. Chinese Patent No. CN204106194U disclosed an opthalmic device attached to a vehicle itself. Chinese Patent No. CN209463962U teaches of a lens-making facility within a vehicle. U.S. Pat. Nos. 6,264,329B1 and 3,201,795A disclosed opthalmic tables for equipment, however it is not designed to be mobile. Chinese Patent No.s CN204201404U disclosed an optometry table with adjustable arm support for opthalmic equipment—however it is not mobile. Most of these instruments are large and contain sensitive lights and precision lenses and no patents in the prior art were found that combined these instruments onto a mobile cart for transport into communities in vehicles.

### SUMMARY OF THE INVENTION

A mobile ophthalmic unit is disclosed. An object of the present invention is to provide a system that supports a multitude of ophthalmic devices on a single desk. For example, the phoropter is installed on an articulating arm that is attached to a metal post. This post is then anchored into the side of the mobile opthalmic unit by means of several cylinder clamps. In this example an ophthalmoscope is also mounted along with its associated controller on the mobile unit. The most common ophthalmic instruments are all within reach of the optometrist while using the unit.

Another object of the invention is to allow users to customize the mobile ophthalmic unit to their assessment needs. For example, one optometrist outfits the unit with a slit lamp and opthalmoscope while another only optometrist outfits the unit with pachymeters and tonometers etc.

Another object of the invention is to allow optometrists to easily install the mobile ophthalmic unit into a cargo van or bus that allows the unit to be transported and used in the field. The unit has locking caster wheels that allow it to be wheeled during installs or removals from such vehicles. Other embodiments of the invention can be installed inside a patient's facility or work place etc.

Another object of the invention is to allow the invention to be secured inside a vehicle to prevent equipment safe while in transport. The mobile ophthalmic unit has anchors that secure the apparatus to a wall inside the vehicle and allow it to be adjusted and affixed to the wall preventing movement.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

FIG. 1 showing a front perspective view of the invention.

FIG. 2 a front perspective view of the invention.

FIG. 3 showing a side perspective view of the invention.

### DETAILED DESCRIPTION OF THE FIGURES

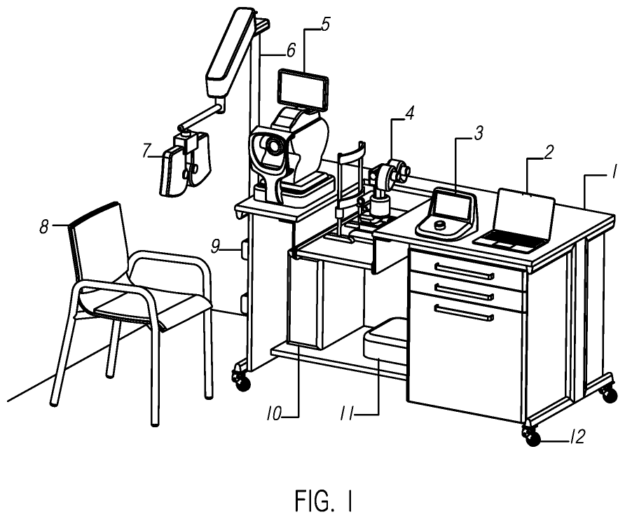

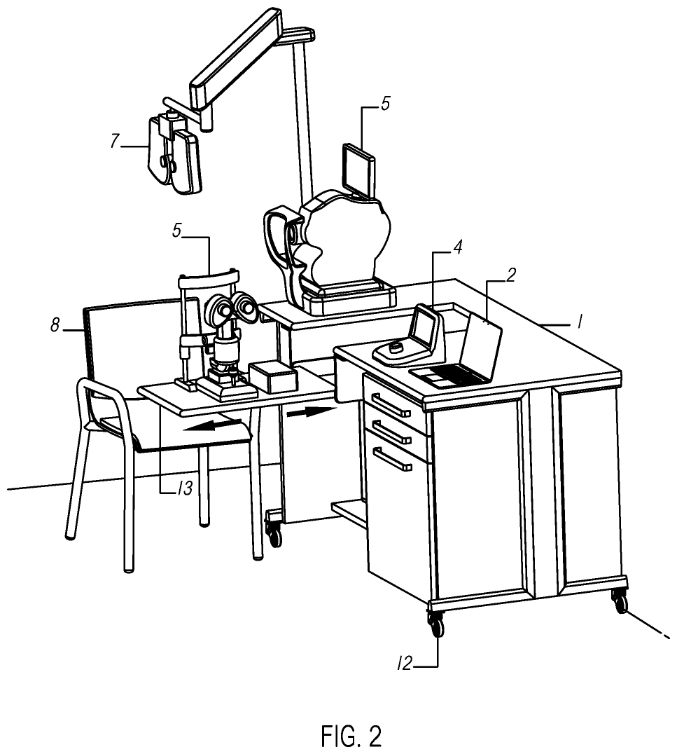

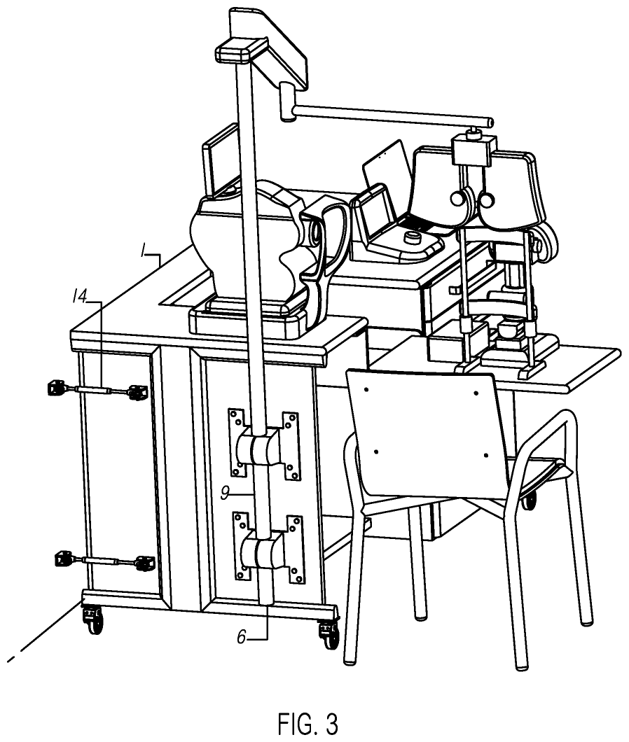

FIG. 1 and FIG. 2 showing a preferred embodiment of the ophthalmic unit 1 comprised of a conventional desk configured to receive equipment such as but not limited to laptop 2, equipment controller 3, slit lamp 4, ophthalmoscope 5, phoropter 7, phoropter post 6, computer 10, printer, and having locking caster wheels 12. The apparatus having slit lamp shelf 13 with extendable and retractable rails on two sides allowing said slit lamp 5 to be positioned in place for a patient sitting in chair 8. Said slit lamp shelf 13 also having a locking mechanism to keep it extended or retracted such as but not limited to wing screws, cam levers etc. FIG. 3 showing ophthalmic unit 1 being selectively affixed to a wall by means of an adjustable anchor device 14 such as but not limited to ball latch clamps, toggle clamps, lead screws, fasteners, etc. The propter post 6 being selectively affixed to the ophthalmic unit 1 by means of but not limited to a fixture 9 comprised of but not limited to compression clamps, swivel clamps being bolted to H-shaped braces, plates and the like. Other peripheral equipment can be stored on ophthalmic unit including but not limited to a laptop computer with a detachable screen, a WiFi router and an internet hotspot and affixed to ophthalmic unit by means of metal strips.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention.

What is claimed is:

1. A mobile ophthalmic unit comprised of the following parts:
a) ophthalmic equipment for treating patients;
b) a desk for supporting ophthalmic equipment;
c) locking caster wheels for moving ophthalmic equipment;
d) metal strips for securing peripheral equipment; and
e) anchor devices for securing ophthalmic equipment to a wall.

2. The mobile ophthalmic unit of claim 1 wherein the equipment for treating patients including phoropter on an adjustable arm, an extendable slit lamp, an ophthalmoscope with controller, laptop computer with a detachable screen, a WiFi router and an internet hotspot.

3. The mobile ophthalmic unit of claim 1 wherein the desk for supporting ophthalmic equipment includes a slit lamp shelf 13 that is selectively extendable and retractable rails.

4. The mobile ophthalmic unit of claim 1 wherein the anchor devices are adjustable.

5. A method for performing on-site eye exams. The method comprised of the following steps:
a) securing ophthalmic equipment;
b) installing the mobile ophthalmic unit in a vehicle;
c) securing the mobile ophthalmic unit to a wall; and
d) adjusting ophthalmic equipment for a patient.

6. The method for performing on-site eye exams wherein the securing ophthalmic equipment includes installing the ophthalmic equipment to the desk using the metal strips of claim 1.

7. The method for performing on-site eye exams wherein the installing the mobile ophthalmic unit in a vehicle is performed using the locking caster wheels of claim 1.

8. The method for performing on-site eye exams wherein the securing the mobile ophthalmic unit to a wall is performed using the anchor devices of claim 1.

9. The method for performing on-site eye exams wherein the adjusting ophthalmic equipment for a patient includes extending the slit lamp shelf of claim 3.
